(19) United States
(12) Patent Application Publication
(10) Pub. No.: US 2005/0172366 A1
(43) Pub. Date: Aug. 4, 2005

(54) Method for corn seed sizing

(76) Inventor: Jonathan Carl Popp, Gilbert, IA (US)

Correspondence Address: POPP ENGINEERING, INC., P.O. BOX 1871, 2710 FORD ST, AMES, IA, 50010, US

(21) Appl. No.: 10/313,653

(22) Filed: Dec. 9, 2002

(51) Int. Cl. A01H 5/00; B07B 1/00

(52) U.S. Cl. 800/320.1; 209/235

(57) ABSTRACT

Flat screens are used to size corn seed into sizes separated by width and thickness. By combining these screens into modular machines, multiple sizes can be made inside each machine. By combining several modular machines, two to eight sizes or more can be made in the assembled system. The modular machines have divider pans and multiple collection pans; this creates the flexibility of the system by having different seed flows and number of seed sizes. The movement of the seed across the flat screens is very gentle and reduces mechanical damage to the seed. Assembly of the screens layers and how they are used in the flow of seed is unique.

### CROSS-REFERENCE TO RELATED APPLICATIONS

“Not Applicable”

### STATEMENT REGARDING FEDERALLY SPONSORED RESEARCH OF DEVELOPMENT

“Not Applicable”

### REFERENCE TO SEQUENCE LISTING, A TABLE, OR A COMPUTER PROGRAM LISTING COMPACT DISK APPENDIX

“Not Applicable”

### BACKGROUND OF THE INVENTION

1. This invention pertains to agricultural processes and more particular to the process of sizing corn seed. Corn seed and other seeds have been sized for many years for two basic reasons: purity and plant ability. This sizing has been accomplished by using both flat and cylindrical screens with round and slotted holes. The round holes were used to make a width separation of the seed and the slotted holes were used to make a thickness separation of the seed.

2. Several manufacturers have made the cylinder style of seed sizing machines. This cylindrical design only allows one size to be made per machine. If multiple sizes are required, multiple machines assembled in a fixed pattern are required.

3. One manufacturer named Superior Processing Equipment manufactured a machined called the Rock-It Corn Grader in the 1960's. This machine would have the closest resemblance to the patent application with the following differences. This machine was only available in one size with one size of screens. The screens used were either tapered round holes or v-rib slotted holes. A limited number of different holes sizes wee available for this machine. The screen cleaning mechanism utilized an assembly of traveling rubber bars that tapped the bottom of the screens. This machine was only available with two screen decks with two screen layers in each deck. Each screen was 20 inches wide and 47 inches long (508 millimeters by 1194 millimeters).

4. The Rock-It style of machine is no longer made nor is parts available for the few remaining machines. The Rock-It grader is also considered obsolete due to the high maintenance, slow capacity, and lack of accuracy due to the short screens.

5. The cylinder style of machine crates too much tumbling action which results in mechanical damage to the seed. This damage significantly reduces the ability of the seed to germinate and drops the quality below actable levels for sale. The cylinder style of machine does not have any flexibility and as the seed sizing changes due to differing hybrid characteristics, capacity is reduced and efficiency is reduced due to the machines not running at optimum capacity.

### BRIEF SUMMARY OF THE INVENTION

1. This is a new method for sizing corn seed and other seeds. The system utilizes modular flat screen machines that can be assembled in combinations of 1 to 4 machines. The design and specification of the modules is important to the screen cleaning and sizing efficiency of the system. Each module has multiple screen layers and multiple product flow options. The flexibility of the machines as well as the unique assembly of the modules creates the ability to size the corn seed based on the individual characteristics of the seed lot. This flexibility results in higher sizing accuracy, capacity, higher seed quality, and number of sizes of seed than is currently possible.

### BRIEF DESCRIPTION OF THE DRAWINGS

FIG. 1A: Drawing showing current cylinder sizing machine assembly.

FIG. 2A: Drawing showing flow possibilities in a two shoe, two screens per shoe, screening module.

FIG. 2B: Drawing showing flow possibilities in a two shoe, three screens per shoe screening module.

FIG. 2C: Drawing showing flow possibilities in a two shoe, four screens per shoe, screening module.

FIG. 3A: Drawing showing seed sizing flow utilizing two modules.

FIG. 4A: Drawing showing seed sizing flow utilizing three modules.

FIG. 5A: Drawing showing seed sizing flow utilizing four modules.

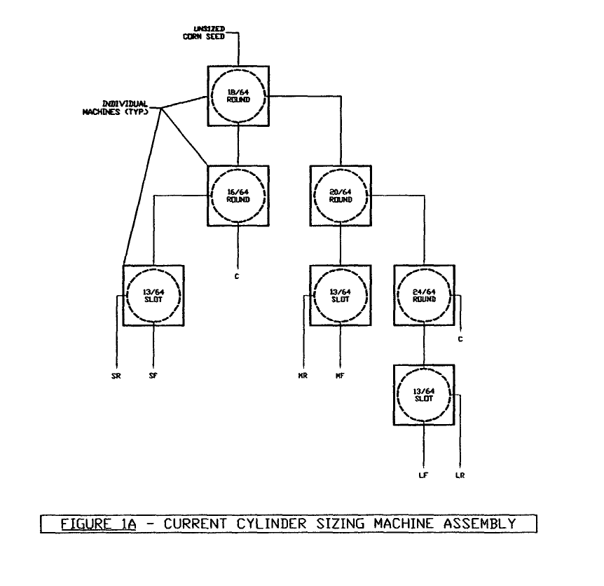

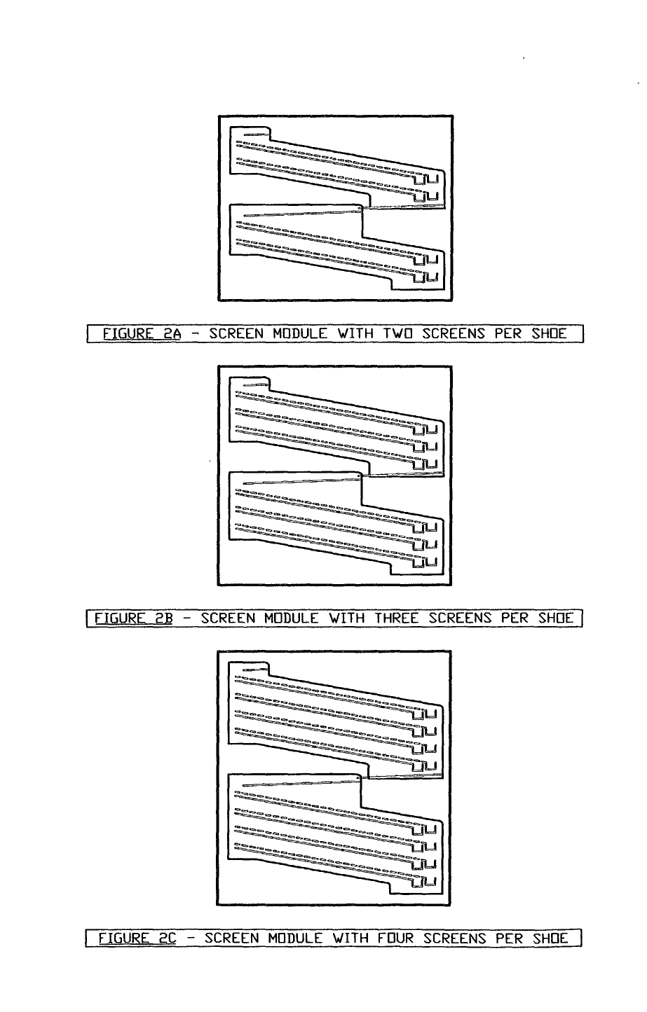

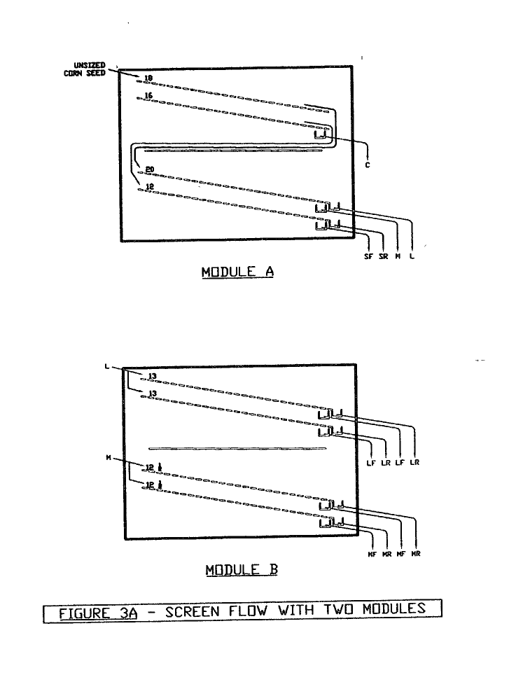

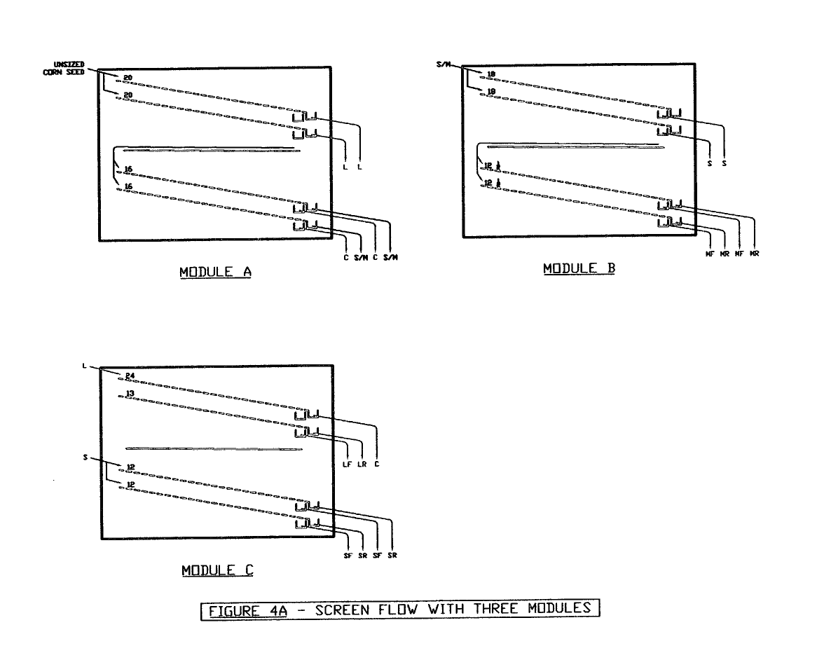

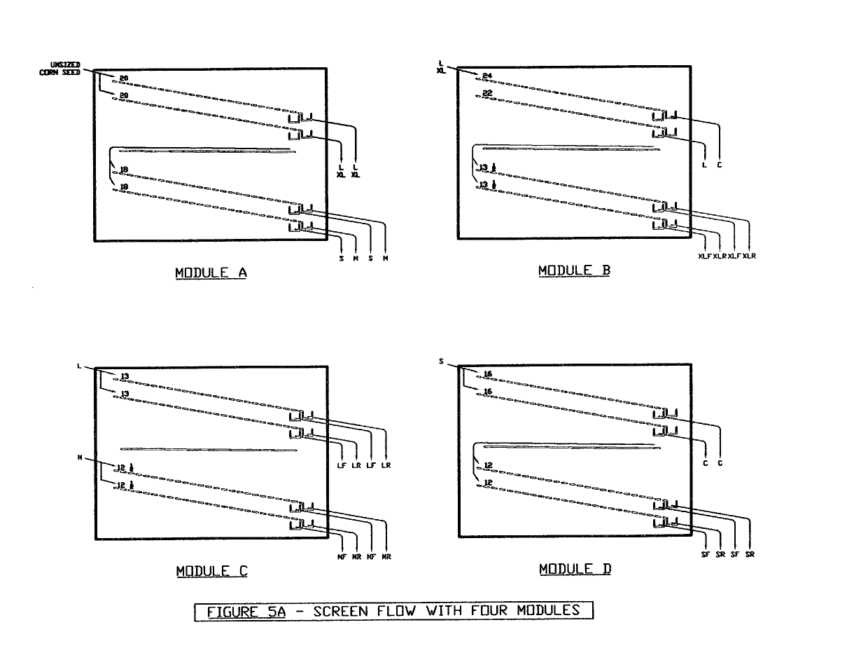

### DETAILED DESCRIPTION OF THE INVENTION

1. This method is for the assembling a system of flat screen modules for the sizing of corn seed and other seeds by differences in seed thickness and width. The results of this method of seed sizing are more sizing flexibility, higher system capacity, and higher seed quality. The drawings show several different sizing flows that are possible with this method. Since corn seed and other seed types that would use this system are an agricultural product, the seed changes from year to year, among differing growing locations, and among differing seed genetics.

2. This method of seed sizing requires a screen modular machine that has specific characteristics to be effective and capture the benefits of the system. Currently there are only two manufacturers that build machines that have these specifications. The manufacturers are Damas A/S located in Faaborg, Denmark and Westrup A/S located in Slagelse, Denmark. The machine needs to have the following specifications: adjustable feeding system to accurately meter the seed across the full width of the screens, shoes that will hold several screens and be accessible to allow for ease of screen changes, a drive system that will shake the screens in a reciprocating manner at a speed of 250 to 330 motions per minute and a distance of 1 3/16 inch (30 millimeters), screens sloped in the direct of seed flow of 2 to 8 degrees. The screens need to be cleaned and vibrated with rubber balls that are of a specific compound, size, and compartment size. The screens need to be punched and assembled in a particular way and of a particular material.

3. The arrangement of the screens is one of the most important features of the modular machine. By having the ability to have multiple screen layers arranged in multiple flow patterns, the operator can adjust the machine to fit the needs of the seed. This fitting of the modules to the seed is significantly different to all existing seed sizing systems. Each screen layer needs to have the material from the screen to be collected in removable chutes. This allows the discharges of the screens to be kept separate and directed to differing locations. Since each screen shoe can have differing numbers of screen layers, dividers to allow the screens to be used in parallel or series flow need to be used for this flexibility.

4. The more modules and the more screens in each shoe of the modules the higher the capacity of the system and the greater the number of potential sizes. The flow of the seed through the screens and the locations of the collection chutes allow this to occur. This flexibility feature of the method allows the system to be adjusted to match the characteristics of the particular seed due to the variability of this agricultural product.

5. Each location that is sizing corn seed or other seed types will need to determine their particular needs to select how to best assemble the modules to meet their sizing characteristics and capacity requirements. From this information the basic system will be assembled and the operator will utilize the flow options of the modules to change the system to meet differing seed requirements. Due to the modular design of the machines, how they are installed is also flexible in regards to location of the machines, location of the holding bins, and the size and location of the material handling or spouting units.

6. The standard size modular machine is 134 inches (3400 millimeters) long by 77 inches (1956 millimeters) wide by 94 1/2 inches (2400 millimeters) tall.

Thus, it is apparent that there has been provided, in accordance with the invention a method that fully satisfies the aims and advantages listed. While this method has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims:

1. Corn seed is produced and sized for sale to farmer growers for planting to produce corn plants and resultant grain.

2. The seed in claim 1 must germinate to produce a viable plant.

3. The seed in claim 1 is sized for plantability, purity of type, and to improve the performance of secondary conditioning equipment.

4. The seed in claim 1 is sized by width using round hole screens and by thickness using slotted hole screens.

5. The seed in claim 4 currently is sized utilizing cylinder screens available in sizes that range in increments of 0.5/64 s of an inch. This means that there is a different screen size available every 0.20 millimeters. This new method allows use of flat screens that are available in size increments of 0.10 millimeters. This availability increases the sizing flexibility and accuracy.

6. The screens listed in claim 5 are made of material that is cold rolled low carbon steel that has an open area of 40 percent and a metal thickness of 20 gauge (0.0359 inches or 0.091 millimeters). This material to conform to ASTM A366 requirements. The material may also be galvanized and the thickness will then be 0.0396 (0.100 millimeters) and conform to ASTM A525 requirements.

7. A modular machine with multiple screens installed in one or more shoes holds the screens in claim 5.

8. The modular machine in claim 7 has multiple pans and discharge chutes that can be added or removed to allow the screens listed in claim 5 to allow the screens to be used in parallel or series product flow.

9. The modular-machine in claim 7 is to have a reciprocating drive system that creates a back and forth motion with a travel distance of 1 3/16 inches (30 millimeters). This drive system should be adjustable in speed from 250 to 330 motions per minute (RPM).

10. The modular machine in claim 7 is to have an adjustable feeding system that allows the seeds to be fed at varying rates in a uniform manner across the entire screen width.

11. The modular machine in claim 7 is to utilize a rubber ball screen cleaning and vibrating system. Rubber balls of 1 to 1.25 inches (25 to 30 millimeters) are to be contained in multiple compartments located under the screens listed in claim 5. The compartments are to be no larger than 12 inches by 6 inches (300 millimeters by 150 millimeters). The rubber balls are to be made of material to give adequate bounce under temperature conditions the machine is operating.

12. The modular machine listed in claim 7 is to have the screens installed in a downward sloping angle in the direction of seed travel of between 2 to 8 degrees. It is helpful to have this angle adjustable but not imperative.

13. The modular machine listed in claim 7 is combines with two or more modular machines of like kind to create a sizing system to make from two to 10 seed sizes. The installation of these modules needs to be in a way that by spouting or additional material handling equipment the seed sizes from the screen chutes listed in claim 8 can be transferred to other parts of the preceding ore succeeding modules.

14. The differing seed sizes created by the assembly of modular machines listed in claim 13 needs to be collected in individual holding bins or other types of vessels for additional conditioning.

15. Some of the seed sizes created in the system listed in claim 13 may need to have additional sizing completed. The system will have a way to return the seed to the modular machine or machines. The screens will need to be changed when the additional sizing is performed.

16. The screens listed in claim 5 and used in the modular machine listed in claim 7 and assembled as listed in claim 13, are available in several width to accomplish differing capacity of the machines. The total length of the screens is 94 1/2 inches (2400 millimeters). This total length is normally made up by using three sections to allow the screens to be handled and changed. The total length of screens is important to the efficiency of the screen separation.

17. The rubber balls listed in claim 11 create a vibration in the screens listed in claim 6 to help the seed find and move through the differing hole sizes. This vibration increases the accuracy and the capacity of the screens.

18. The quality of the seed is improved in this system as listed in claim 13 over cylinder styles of sizing due to the flow of the seed across the perforated screens and the elimination of the tumbling of the seed as found in a cylinder style of machine.

19. The quality of the seed is improved in this system as listed in claim 13 over cylinder styles of sizing due to the flow of the seed across the perforated screens and the elimination of the sharp edges of the screen material found in a cylinder style of machine.
